(19) United States
(12) Patent Application Publication
(10) Pub. No.: US 2009/0197347 A1
(43) Pub. Date: Aug. 6, 2009

(54) IMMUNOASSAY FOR PLASMODIUM FALCIPARUM AND ASSAY DEVICE USED THEREFOR

(75) Inventors: Mi Jin Sohn, Daejeon (KR); Seung Bum Yoo, Daejeon (KR); Yeon Chul Kim, Daejeon (KR); Jae Hoon Oh, Daejeon (KR); Eunkyung Kim, Gyeonggi-do (KR); Seung Ho Choo, Daejeon (KR)

Correspondence Address: CANTOR COLBURN, LLP, 20 Church Street, 22nd Floor, Hartford, CT, 06103, US

(73) Assignee: LG LIFE SCIENCES, LTD., Seoul (KR)

(21) Appl. No.: 11/571,335

(22) PCT Filed: Jun. 28, 2005
(86) PCT No.: PCT/KR2005/002010

§ 371 Date: Dec. 27, 2006

(30) Foreign Application Priority Data
Jun. 30, 2004 (KR) 10-2004-0049985

(51) Int. Cl. G01N 33/569; G01N 33/566

(52) U.S. Cl. 436/501

(57) ABSTRACT

Disclosed are an immunoassay of Plasmodium falciparum for determining the presence/absence of a specific and/or antibody thereof via label in conjugates bound to the specific antigen and/or antibody present in a sample, comprising immobilizing the specific antigen and antibody of Plasmodium falciparum on a solid phase, adding a sample obtained from a subject of interest to the solid phase so as to induce specific antibody-antigen reaction, adding a conjugate of the antigen and a label and a conjugate of the antibody and a label, separately prepared, so as to induce binding of at least one of the conjugates; and an assay device comprising the above-mentioned solid phase and conjugates.

The present invention can effect specific detection of antigens and/or antibodies in patients with manifested malaria-symptoms as well as malaria carriers and can also be efficiently employed in samples at the early stage of malaria infection that is difficult to detect via conventional arts. Further, due to the capacity to utilize sera and blood plasma rather than whole blood, the present invention is well suited to large-scale examination such as blood screening.

### FIELD OF THE INVENTION

The present invention relates to an immunoassay and diagnostic reagent for Plasmodium falciparum and an assay device used for the same. More specifically, the present invention relates to an immunoassay for detecting specific antigens and/or antibodies of Plasmodium falciparum in blood sample, comprising immobilizing specific antigen(s) of Plasmodium falciparum such as a heat-shock protein 70, a merozoite surface protein and a glycophorin binding protein 130 in combination with a specific antibody prepared utilizing a histidine-rich protein II as the antigen, on a solid phase adding a sample obtained from the subject of interest to the solid phase so as to induce reaction between the antigen and the antibody specific for the antigen, and adding a antigen conjugate and a antibody conjugate, separately prepared, so as to induce binding of at least one of the conjugates; and an assay device comprising the above-mentioned solid phase and conjugates.

### BACKGROUND OF THE INVENTION

Malaria is a fatal disease caused by infection of human red blood cells with mosquito-borne malaria parasites. 100 million people worldwide live in dangerous regions that may become exposed to high risk of malaria infection. Approximately 50 million people are affected by malaria and more than 2 million people die from it every year. Malaria was present throughout the globe in the past, but some regions have showed reduced or no morbidity due to malaria since 1960's, due to effective disease control. However, malaria is now reemerging due to abnormal weather patterns such as El Nino, increase in drug-resistant bacteria, increased resistance to insecticides, and the like.

Malaria is caused by malaria parasites of the genus Plasmodium. Four species of Plasmodium are known to produce the disease: Plasmodium vivax, Plasmodium falciparum, Plasmodium malariae and Plasmnodium ovale. Among these, Plasmodium falciparum is a fatal pathogen exhibiting the most potent toxicity and very high mortality, thus causing the deaths of considerable numbers of malaria-infected patients. Nonetheless, there has been no development of diagnostic reagents having high sensitivity and specificity, and suitable for blood screening, and thus blood smearing via microscopic observation is prevalently employed to detect the presence of malaria pathogens.

Among currently developed diagnostic reagents for Plasmodium falciparum, a diagnostic reagent directed to an antigen is based on an enzyme immunoassay which detects malaria-specific antigens utilizing antibodies against HRP II (histidine-rich protein II) or LDH (lactate dehydrogenase). This method has an advantage in that it enables direct examination of malaria parasites, but suffers from disadvantages such as lower sensitivity than blood smearing, thus failing to effectively detect pathogens in patients harboring few members of malaria parasites or patients under a latent period. In addition, some of the currently used antigen-diagnostic reagents are designed to detect malaria parasites present in red blood cells, and thereby are incapable of taking advantage of sera or blood plasma as a target sample. Therefore, a separate step of disrupting red blood cells is necessary, which is thus not suitable for examination such as blood screening.

Meanwhile, a diagnostic reagent for a Plasmodium falciparum antibody is a method of detecting the antibodies rather than malaria parasites, and therefore is advantageously capable of detecting pathogenic parasites even when patients carry a very few number of parasites under the latent period. However, this method suffers from difficulty to male accurate diagnosis in patients during the early stage of infection, due to the presence of a window period prior to production of antibodies following malaria infection. Further, the currently developed diagnostic reagents for Plasmnodium falciparum antibody take advantage of an indirect enzyme immunoassay utilizing the cell homogenates that are primarily obtained by culture of malaria parasites, and thus are known to show poor specificity and sensitivity (Vox Sanguinis, 1999; 77: 237-238). Meanwhile, a great deal of efforts has been made to develop a label for diagnosing antibodies against Plasmodium falciparum, but there are few known label antigens useful for antibody diagnosis.

### SUMMARY OF THE INVENTION

Therefore, the present invention has been made to solve the above problems, and other technical problems that have yet to be resolved.

Specifically, it is an object of the present invention to provide an immunoassay for determining the presence of Plasmodium falciparum-specific antigens and/or specific antibodies in samples such as blood plasma, sera and body fluid, obtained from the subject of interest, by simultaneously using specific antigen(s) and a specific antibody of Plasmodium falciparum. The immunoassay in accordance with the present invention exhibits higher sensitivity and specificity as compared to conventional methods and thus is capable of diagnosing not only malaria patients, but also malaria carriers. In addition, due to use of samples such as blood plasma and sera, the immunoassay in accordance with the present invention can be very usefully applied to diagnosis of Plasmodium falciparum including blood screening.

Another object of the present invention is to provide an assay device that can be used in the above-mentioned immunoassay. Despite the simplified constitution, the assay device of the present invention may be implemented as a diagnostic agent, diagnostic kit, and the like, for confirming infection with Plasmodium falciparum.

### DETAILED DESCRIPTION OF PREFERRED EMBODIMENTS

In accordance with an aspect of the present invention, the above and other objects can be accomplished by the provision of an immunoassay for determining the presence of specific antigens and/or antibodies of Plasmodium falciparum via labels in conjugates bound to the specific antigen and/or antibodies present in a sample, comprising:

immobilizing the specific antigen(s) and specific antibody of Plasmodium falciparum on a solid phase

adding a sample obtained from the subject to be examined to the support to induce reaction between the antigen and the antibody specific for the antigen; and

adding a conjugate of the antigen and a label (“antigen conjugate”) and a conjugate of the antibody and label (“antibody conjugate”), separately prepared, so as to induce binding of at least one of the conjugates.

Therefore, in accordance with the present invention, due to simultaneous use of the specific antigen and antibody of Plasmodium falciparum, it is possible to confirm the presence of antigens or antibodies even when either antigen or antibody is only present in the target sample. Such a method is a novel method that had never been disclosed or proposed by conventional arts, and the present inventors have confirmed through various extensive experiments that the present method provides unexpected synergistic effects surpassing simple combination of a method using the specific antigen only and a method using the specific antibody only. Details will be described in the following examples (experimental examples and comparative examples).

Preferably, the specific antigen of Plasmodium falciparum is selected from the group consisting of a heat-shock protein 70 (HSP 70), a merozoite surface protein (MSP) and a glycophorin binding protein 130 (GBP 130) of Plasmodium falciparum and any combination thereof.

Preferably, the specific antibody of Plasmodium falciparum may be an anti-histidine-rich protein II (anti-HRP II), and for example, may be monoclonal or polyclonal antibodies derived from animals or antibodies derived from genetic recombination techniques.

Particularly preferably, HSP 70, MSP and GBP 130 are used in combination as specific antigens, and anti-HRP II is used as the specific antibody.

In the present invention, particularly in the following examples, the above-mentioned antigens are obtained by isolating RNAs from red blood cells of patients infected with Plasmodium falciparum, constructing corresponding cDNAs of HSP 70, MSP, GBP 130 and HRP II antigens, respectively, utilizing genetic recombination, and culturing the obtained cDNAs in transformed Escherichia coli, followed by isolation and purification. In addition, anti-HRP II, which is an antibody specifically responding to HRP II, is obtained by injecting the HRP II to rabbits in conjunction with an immune adjuvant, isolating and purifying polyclonal antibodies that are anti-HRP II sera to separate only pure antibody fractions. However, this method is only the illustrative example of the present invention and therefore it will be apparent to those skilled in the art that the above-mentioned antigens and antibodies may be prepared via use of various other methods well known in the related art.

Immobilizing the specific antigens and specific antibodies of Plasmodium falciparum onto the solid phase, respectively, may be carried out by various methods. For instance, an immobilization process may be carried out by adding these specific antigens and antibodies to be attached on the solid phase, followed by re-addition of a blocking agent containing bovine serum albumin.

The solid phase used in the immunoassay of the present invention is a solid material capable of stably immobilizing the specific antigens and specific antibodies of Plasmodium falciparum without causing nonspecific reactions. For example, mention may be made of vessels, membranes and particulate matters composed of glass, synthetic resins, semi-synthetic resins or metal materials. Specifically, synthetic resin well plates made of polystyrene, membranes made of nitrocellulose or nylon, glass, gold particle-deposited particulate matters or the like may be used.

The labels in the antigen conjugate and antibody conjugate are not particularly limited, so long as they are capable of confirming the presence of the conjugates bound to antigens and/or antibodies present in samples of interest. For example, the label is selected from the group consisting of horseradish peroxidase (HRP), alkaline phosphatase, β-galactosidase, colloidal gold, fluorescein, dye and any combination thereof.

Although an example of preparing the antigen conjugate and antibody conjugate is illustrated in the following examples, those skilled in the art will appreciate that any other various methods may be employed.

Whether the conjugates were bound to antigens and/or antibodies in the target sample can be confirmed, for example, by adding a substrate that will be catalyzed by the labels in the conjugates and determining whether the substrate was catalyzed or not. For example, upon using a binding agent in which the antigens or antibodies were labeled with horseradish peroxidase (HRP), an enzyme that is utilizable as a chromagen leading to color development, it is possible to confirm whether the conjugates were bound to the antibodies and/or antigens in the sample, by adding the substrate that is catalyzed by HRP and determining color development of the substrate. Examples of such decomposition substrate include, but are not limited to, TMB (tetramethyl benzidine) and the like.

The above-mentioned sample used in the immunoassay of the present invention is, when the subject to be examined was infected with Plasmnodium falciparum, one in which the specific antigens and/or antibodies of Plasmodium falciparum may be present. For example, mention may be made of at least one selected from the group consisting of sera, blood plasma, body fluid and any combination, isolated from blood, stool and urine and/or saliva. Preferably, sera or blood plasma is employed.

In accordance with another aspect of the present invention, there is provided an assay device capable of performing the above-mentioned immunoassay, comprising a solid phase having specific antigens and specific antibodies of Plasmodium falciparum immobilized on the surface thereof, and an antigen conjugate and antibody conjugate.

As used herein, the term “assay device”, as described above, is an implicative expression including assay (or diagnostic) kits, reagents and the like, capable of confirming whether specific antigens and/or specific antibodies of Plasmodium falciparum are present in samples obtained from subjects to be examined. Therefore, the assay device in accordance with the present invention may be variously embodied as the diagnostic kit, diagnostic reagent and the like, for Plasmodium falciparum, where appropriate.

When the label in the conjugate is HRP (horseradish peroxidase), the assay device may further include a color developer such as TMB that is catalyzed by HRP, if necessary. In addition, the assay device may further include a measuring instrument such as a spectrophotometer capable of confirming the color developer catalyzed by HRP.

### BRIEF DESCRIPTION OF THE DRAWINGS

The above and other objects, features and other advantages of the present invention will be more clearly understood from the following detailed description taken in conjunction with the accompanying drawings, in which:

FIGS. 1A and 1B are a plasmid map of pET15fx-HSP70 and an amino acid sequence of HSP70, respectively;

FIGS. 2A and 2B are a plasmid map of pET15fx-MSP19 and an amino acid sequence of MSP19, respectively;

FIGS. 3A and 3B are a plasmid map of pET15fx-GBP130 and an amino acid sequence of GBP130, respectively;

FIGS. 4A and 4B are a plasmid map of pET115fx-HRP II and an amino acid sequence of HRP II, respectively;

FIG. 5 shows the detection results of specific antibodies and antigens of Plasmodium falciparum for samples of normal specimen and malaria (Plasmodium falciparum) patients in experimental example 1, via a method in accordance with the present invention;

FIG. 6 shows the detection results of specific antibodies of Plasmodium falciparum for samples of normal specimen and malaria (Plasmodium falciparum) patients in comparative example 1; and

FIG. 7 graphically shows the detection results of specific antigens of Plasmodium falciparum for samples of normal specimen and malaria (Plasmodium falciparum) patients in comparative example 2.

### EXAMPLES

Now, the present invention will be described in more detail with reference to the following examples. These examples are provided only for illustrating the present invention and should not be construed as limiting the scope and spirit of the present invention.

### Example 1

### Construction and Expression of Recombinant Malaria Antigens

In order to isolate RNAs from Plasmodium falciparum positive blood, a TRI Reagent™ (Sigma, Cat. No. T9424) was used. Isolation was carried out as follows, according to the instructions of a reagent manufacturer.

0.1 ml of malaria positive blood was mixed and reacted with 0.1 ml of TRI Reagent™ (Sigma, Cat. No. T9424) at room temperature for 5 min. The resulting solution was mixed and reacted with 20 ml of BCP (1-bromo-3-chloropropane) at room temperature for 5 min and then centrifuged at a temperature of 4° C. and 12,000 rpm for 15 min. Among 3 layers formed after centrifugation, the upper layer containing RNAs was transferred to a fresh tube and 50 μl of isopropanol was added thereto, and incubated at room temperature for 5 min. The resulting solution was centrifuged at a temperature of 4° C. and 12,000 rpm for 10 min and the supernatant was discarded. Precipitates were washed with 75% ethanol and dissolved in 20 μl of distilled water for use.

9 μl of the thus-purified RNA was mixed and reacted with 1 μl of the constructed N6 random primer (Genotech) at a temperature of 65° C. for 5 min, and the reaction solution was transferred on ice. 4 μl of a RT buffer, 2 μl of 0.1 M DTT, 1 μl of 10 mM dNTP, 1 μl of Rtase (Gibco), 1 μl of an RT inhibitor (Promega) and 1 μl of D.W. (deionized water) were added to the solution and the resulting mixture was reacted at a temperature of 42° C. for 1 hour and then at a temperature of 70° C. for 10 min to construct cDNAs.

Synthesis of cDNA was carried out using a ThermoScript™ RT-PCR System (Invitrogen, Cat No. 11146-024). 5 μl of the above-purified RNA, 1 μl of a random-hexamer, 2 μl of 10 mM dNTP mix, and 4 μl of DEPC-treated water were mixed together, reacted at a temperature of 65° C. for 5 min and transferred on ice. 4 μl of 5×cDNA synthesis buffer, 1 μl of 0.1 M DTT, 1 μl of RNaseOUT™, 1 μl of DEPC-treated water, and 1 μl of ThermoScript™ RT were added to the above solution, were reacted at a temperature of 25° C. for 10 min and then at a temperature of 50° C. for 50 min and incubated at a temperature of 85° C. for 5 min to stop reaction. Thereafter, 1 μl of RNase H was added thereto, the resulting mixture was incubated at 37° C. for 20 min to remove unreacted RNA. The thus-synthesized cDNAs were stored at a temperature of −20° C. and was used as desired.

In addition, in order to carry out PCR, respective primers for 4 antigens were constructed as follows.

(1) HSP 70
Pf-HSP70-NdeI (forward):

(SEQ ID NO:1)

5′-GGATCCCATATGGCTAGTGCAAAAGGTTC-3′
Pf-HSP70-XhoI (reverse):

(SEQ ID NO:2)

5′-GGATCCCTCGAGTTAATCAACTTCTTCAACTGTTGG-3′
HSP70-SphI-5′ (forward):

(SEQ ID NO:3)

5′-AATGGTAAAGAAGCATGCAGATCAATTAAC-3′
HSP70-SphI-3′ (reverse):

(SEQ ID NO:4)

5′-GTTAATTGATCTGCATGCTTCTTTACCATT-3′
(2) GBP130
Pf-GBP130-NdeI (forward):

(SEQ ID NO:5)

5′-GATTATCATATGAGAGAAAGCAGAATTTTAGC-3′
Pf-G130BamHI-5′ (forward):

(SEQ ID NO:6)

5′AGAGAATATGCCGCGGATCCAGAATATCGT-3′
Pf-G130BamHI-3′ (reverse):

(SEQ ID NO:7)

5′-ACGATATTCTGGATCCGCGGCATATTCTCT-3′
Pf-GBP130-XhoI (reverse):

(SEQ ID NO:8)

5′-GGATCCCTCGAGTTATGCTTCGTTATTATCAGC-3′
(3) MSP
Pf-MSP19-NdeI (forward):

(SEQ ID NO:9)

5′-GCAACACATATGGCAGTAACTCCTTCCGTAATTGA-3′
Pf-MSP19-XhoI (reverse):

(SEQ ID NO:10)

5′-GTGTTGCTCGAGTTATAACATACCTTGCAAGTTTCC-3′
(4) HRP II
Pf-HRP II-NdeI (forward):

(SEQ ID NO:11)

5′-TTGTTACATATGAGCAAAAATGCAAAAGGACTT-3′
Pf-HRP II-XhoI (reverse):

(SEQ ID NO:12)

5′-ATAAATCTCGAGTTAATGGCGTAGGCAATGTGT-3′

PCR was carried out using the thus-synthesized cDNAs as a template, and the thus-prepared primers. The PCR reaction was carried out as follows: 10 μl of a Thermopol buffer, 2 μl of 10 mM dNTP, 2 μl of primer 5, 2 μl of primer 3, 2 μl of DNA template, 81 μl of D.W., 1 pt of Vent DNA Polymerase (NEB, Cat. No. M0254L) were charged to a reaction vessel. After standing at 95° C. for 2 min, 40 cycles of reaction at 95° C. for 40 sec, 55° C. for 30 sec and 72° C. for 2 min were repeated and then incubated at 72° C. for 10 min. The amplified DNAs were confirmed by electrophoresis on 1% agarose gel.

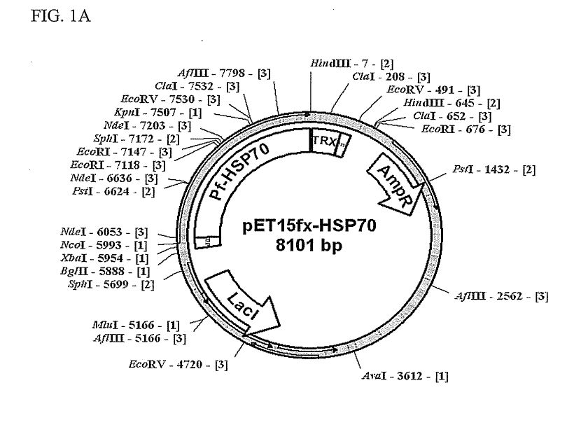

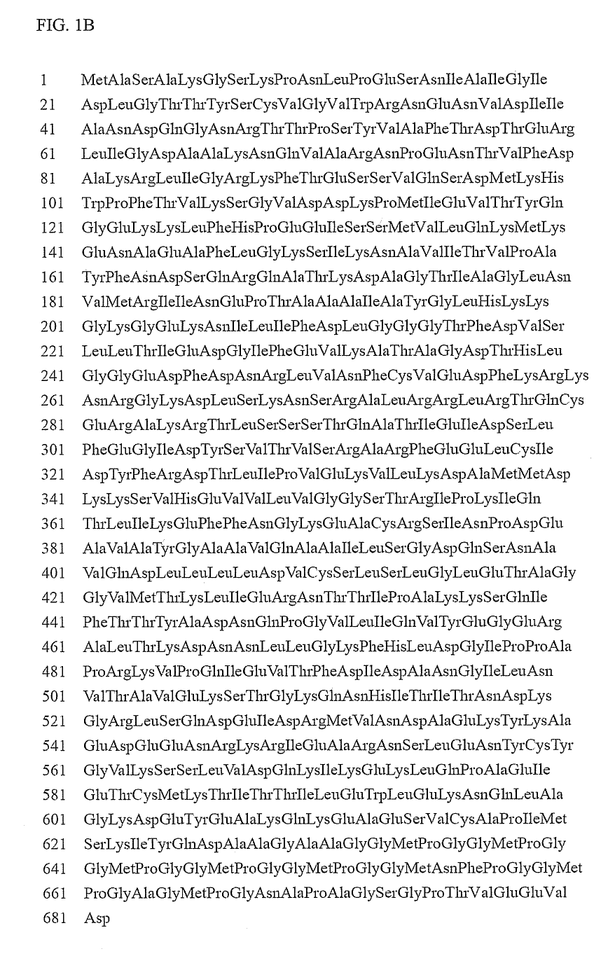

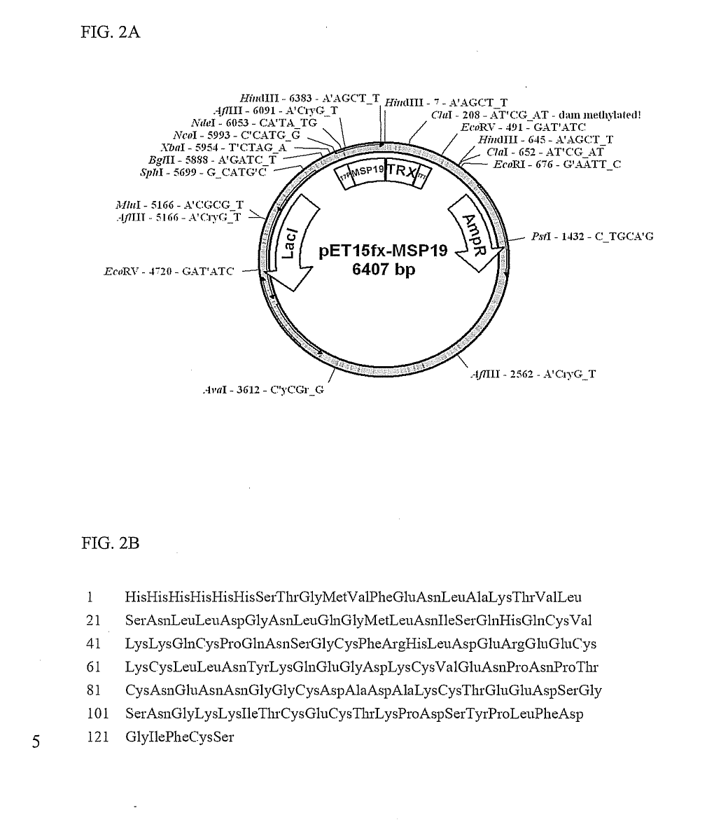

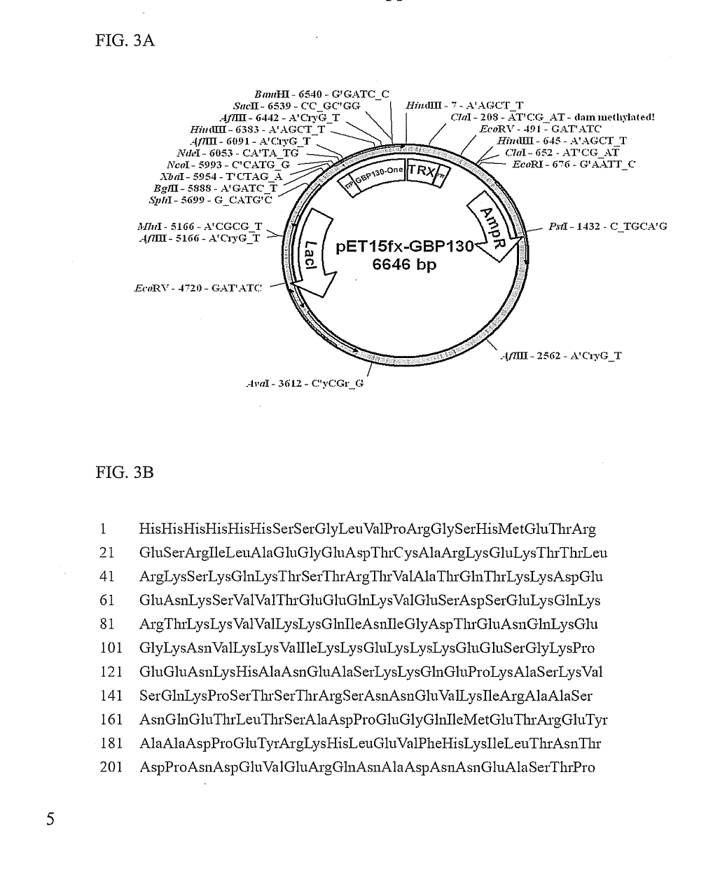

PCR reaction products were separated using a Power Gel Extraction Kit (BioProgen, Cat. No. 23103). An N-terminal DNA fragment was ligated into pBluescript II KS+(Stratagene, Cat. No. 212207) which had been cleaved with a restrict enzyme EcoRV, using T4 DNA ligase and the resulting ligation product was transformed into E. coli HB101. For a C-terminal, the PCR reaction products were purified and Taq polymerase was added and reacted with the purified C-terminal fragment at a temperature of 72° C. for 10 min. The reaction product was separated using a Power Gel Extraction Kit and ligated into a pGEM-T Easy Vector (Promega, Cat. No. A1360) that was then transformed into E. coli HB101. Respective vectors and inserts were reacted and cleaved with SphI and XhoI at 37° C. for 2 hours. Two restriction fragments were ligated using a T4 DNA ligase and the ligation product was transformed into E. coli HB101. Using T4 DNA ligase, the resulting transformant was ligated into pET15fx, which is a modified version of pET-15b (Novagen, Cat. No. 69661-1) having a conserved histidine residue tag sequence, after cleaving with NdeI and SalI, thereby obtaining a recombinant expression vector. The prepared expression vector was transformed into E. coli BL21(DE3) (Novagen, Cat. No. 69387-3) via use of a calcium chloride method. Expression vectors containing these genes were determined to have amino acid sequences (SEQ ID NOS: 13 through 16), as shown in FIGS. 1 through 4, respectively.

### Example 2

### Purification of Malaria Antigens Expressed in E. coli Transformants

E. coli transformants constructed in example 1 were cultured in an LB medium to which antibiotics ampicillin (100 μg/ml) and chloramphenicol (50 μg/ml) were added for 12 hours. 50 ml of the culture was inoculated again onto 1 L of the LB medium and incubated at a temperature of 37° C. for 2 hours. Cells were grown to an optical density at 600 nm (OD600) of 0.3 and then, incubated to for additional 7 hours by addition of IPTG (isopropyl β-D-thiogalactopyranoside) to a final concentration of 0.2 mM. The culture was centrifuged to separate the cells and the separated cells were suspended in 30 ml of phosphate buffer, homogenized using a Sonicator (Sonifier 450, Branson) and centrifuged at 12,000 rpm for 30 min. Since some portion of HSP 70 protein expressed in E. coli transformants was present in the supernatant, while the remainder was present in centrifuged precipitates, the supernatant and precipitates were separately pooled and were electrophoresed to locate the position of the desired proteins. Among them, the precipitates were completely suspended in a 8 M urea buffered solution (10 mM Tris pH 8.0, 0.5 M sodium chloride, 8 M urea), and centrifuged again to pool supernatant only.

In order to purify HSP 70, MSP, GBP 130 and HRP II expressed from E. coli transformants, these proteins were adsorbed onto a ProBond column (Invitrogen), taking advantage of histidine residues labeled at the N-terminus of proteins. For supernatant of the first cell homogenate, 10 mM Tris (pH 7.5), 0.5 M sodium chloride, and 5 mM imidazole were employed as equilibrium buffer. For the second supernatant dissolved in 8 M urea, proteins were adsorbed onto the column using equilibrium buffer to which 6 M urea was added. Non-adsorbed impurities were removed with buffer containing 20 mM imidazole. Surface proteins adsorbed onto the column were separated by eluting with equilibrium buffer containing 1 M imidazole for the first supernatant, and with equilibrium buffer containing 0.3 M imidazole dissolved therein for the supernatant dissolved in 8 M urea.

### Example 3

### Production and Purification of Anti-HRP II Antibodies

A mixed solution of HRP II antigen purified in example 2 and an immune adjuvant (Sigma, Complete Freund Adjuvant) was administered to rabbits, 3 times at 3-week intervals by intramuscular injection, and blood was collected from rabbits and centrifuged to obtain sera. 45% ammonium sulfate was added to the sera to cause precipitation of antibodies which was then centrifuged at 12,000 rpm for 40 min. The precipitates were dissolved with phosphate buffer and purified by Protein G column chromatography.

### Example 4

### Preparation of Antigen Conjugate and Antibody Conjugate

Recombinant MSP, HSP 70 and GBP 130 antigens, isolated and purified according to the procedure of example 1, were respectively dialyzed to a volume ratio of more than 1:200 at 4° C. for 2 days using 1 L of 0.01 M sodium carbonate buffer, pH 9.6, with exchange of the buffer three times. In addition, for activation of horseradish peroxidase (HRP) that is to be conjugated to MSP, HSP 70, GBP 130 and anti-HRP II, 2 mg of HRP was dissolved in 2 ml of distilled water and 100 μl of 42 mg/ml sodium periodate (NaIO₄) was added to the HRP solution which was then reacted while shaking in a tube wrapped with foil at room temperature for 30 min, thereby oxidizing HRP. When HRP was sufficiently oxidized, 60 μl of 1 M glycerol was added to the reaction solution that was reacted while shaking in a tube wrapped with foil at room temperature for 30 min, thereby terminating oxidization of HRP.

For conjugation between HRP and respective MSP, HSP 70, GBP 130 and anti-HRP II, the above HRP reaction solution was dialyzed against 0.01 M sodium carbonate buffer (pH 9.6) to remove salts. MSP, HSP 70, GBP 130 and anti-HRP II solutions were respectively added to 0.5 ml of the thus-oxidized HRP reaction solution, and the mixtures were then reacted while shaking in a tube wrapped with foil at room temperature overnight, thereby preparing MSP-HRP, HSP 70-HRP, GBP 130-HRP and anti-HRP II-HRP conjugates, respectively. In order to stabilize antigen- and antibody-HRP conjugates after completion of conjugation reaction between HRP and respective antigens and antibodies, 40.8 μl of 4 mg/ml NaBH₄ was added and then reacted while shaking in a tube wrapped with foil at a temperature of 4° C. for 2 hours. Thereafter, the resulting products were dialyzed against 10 mM Tris (pH 7.5) buffer three times to a volume ratio of more than 1:200.

### Experimental Example 1

### Simultaneous Detection of Specific Antibodies and Antigens of Plasmodium falciparum in Accordance with the Present Invention

In order to confirm malaria infection via simultaneous detection of antigens and antibodies of Plasmodium falciparum in accordance with the present invention, the following experiments were carried out.

Solution containing MSP, HSP 70 and GBP 130 in a concentration of 0.1 to 1.0 μg/ml, respectively and rabbit anti-HRP 11 prepared in example 3 in a concentration of 5 to 10 μg/ml were diluted in 0.1 M carbonate buffer (pH 9.5) and then 100 μl of diluted solution was dispensed into each well of a well plate. The well plate was sealed and incubated at room temperature for 18 hours such that the antigens were bound to the well plate. The solution remaining not adhered to the well plate was removed and then 300 μl of a phosphate buffer containing 0.5% bovine serum albumin was added into each well and incubated at room temperature for 6 hours. The solution remaining in the wells was removed and dried, placed in a sealed bag containing a desiccant, which was then stored in a low-temperature refrigerator at 4° C.

100 μl of sample diluent containing 1% BSA (bovine serum albumin), PBS (phosphate buffered saline) and Triton X-100 was added to each well and then 50 μl of sample was added and mixed well. The mixture in the well plate was reacted for 60 min in a incubator. After completion of reaction, the well plate was washed with 300 μl of PBS containing 0.05% Tween 20 five times.

Antigen-HRP conjugates and anti-HRP II-HRP conjugate prepared in example 4 were diluted with a casein/PBS diluent containing Tween 20. 100 μl of the diluted solution was added to each well and reacted 37° C. for 30 min. After completion of reaction, the well plate was washed with 0.05% Tween 20/PBS five times, and 100 μl of a substrate solution containing 100 μg/ml of TMB (tetramethyl benzidine), 0.006% hydrogen peroxide and citric-phosphate buffer (pH 4.5) was added to each well and color development was carried out in a dark room for 30 min. 100 μl of stop solution (1N sulfuric acid) was added to each well to terminate color development reaction, and optical density (OD) at 450 nm was measured with a 96-well plate reader (Molecular Devices), using 650 nm as a reference wavelength. Since the color developer, TMB, in the substrate solution was catalyzed by the HRP conjugate bound to the antibodies, thereby causing a color development reaction, the presence or absence of malaria-specific antigens and antibodies and intensity of antigens and antibodies were detected by measuring the degree of color development in terms of optical density. Samples used in the above experiments are as follows.

(1) Samples of 264 sera from normal specimen.
(2) Samples of 21 sera from Plasmodium falciparum-infected patients in India where people are susceptible to high risk of malaria infection.
(3) Samples of 20 sera from Korean patients infected with Plasmodium falciparum after visiting high-risk regions of malaria infection.

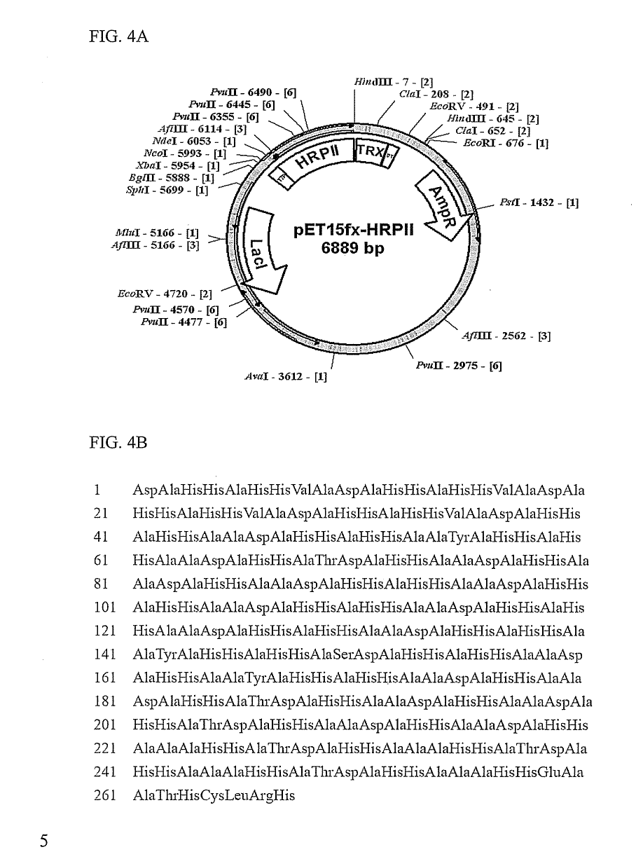

The cut-off value was set as an average absorbance of negative samples plus 0.3. As experimental results, the examined sera were all tested negative upon examining samples of 264 sera from normal specimen. Upon examining 21 samples from Plasmodium falciparum-infected patients in India, it was found that 20 samples (95% sensitivity) were tested positive. For 20 samples from Korean patients infected with Plasmodium falciparum, it was found that 18 samples (90% sensitivity) were tested positive. The results thus obtained are graphically shown in FIG. 5.

### Comparative Example 1

### Detection of Specific Antibodies of Plasmodium falciparum Via Enzyme Immunoassay

For comparison with a method in accordance with the present invention, the method for enzyme immunoassay detecting specific antibodies of Plasmodium falciparum only was carried out according to the following experiments.

Solution of MSP, HSP 70 and GBP 130 were respectively diluted to concentrations of 0.1 to 1.0 μg/ml in a 0.1 M carbonate buffer (pH 9.5) and 100 μl of the diluted solution was added into each well of a well plate. The well plate was sealed and incubated at room temperature for 18 hours such that the antigens added into each well were bound to the well plate. The solution remaining not adhered to the well plate was removed and then 300 μl of PBS containing 0.5% BSA was added into each well and incubated at room temperature for 6 hours. The solution remaining in the wells was removed and dried, placed in a sealed bag containing a desiccant, which was then stored in a low-temperature refrigerator at 4° C.

100 μl of sample diluent containing 1% BSA (bovine serum albumin), PBS (phosphate buffered saline) and Triton X-100 was added to each well and then 50 μl of sample was added and mixed well. The mixture in the well plate was reacted for 60 min in a incubator. After completion of reaction, the well plate was washed with 300 μl of PBS containing 0.05% Tween 20 five times.

Antigen-HRP conjugates prepared in example 4 were diluted with a casein/PBS diluent containing Tween 20. 100 μl of the diluted solution was added to each well and reacted 37° C. for 30 min. After completion of reaction, the well plate was washed with 0.05% Tween 20/PBS five times, and 100 μl of a substrate solution containing 100 μg/ml of TMB, 0.006% hydrogen peroxide and citric-phosphate buffer (pH 4.5) was added to each well and color development was carried out in a dark room for 30 min. 100 μl of stop solution (1N sulfuric acid) was added to each well to terminate color development reaction, and optical density (OD) at 450 nm was measured with a 96-well plate reader, using 650 nm as a reference wavelength. Since the color developer, TMB, in the substrate solution was catalyzed by the HRP conjugates bound to the antibodies, thereby causing color development reaction, the presence or absence of malaria-specific antibodies and intensity of antibody presence were detected by measuring the degree of color development in terms of optical density. Samples used in the above experiments were the same as those of experimental example 1.

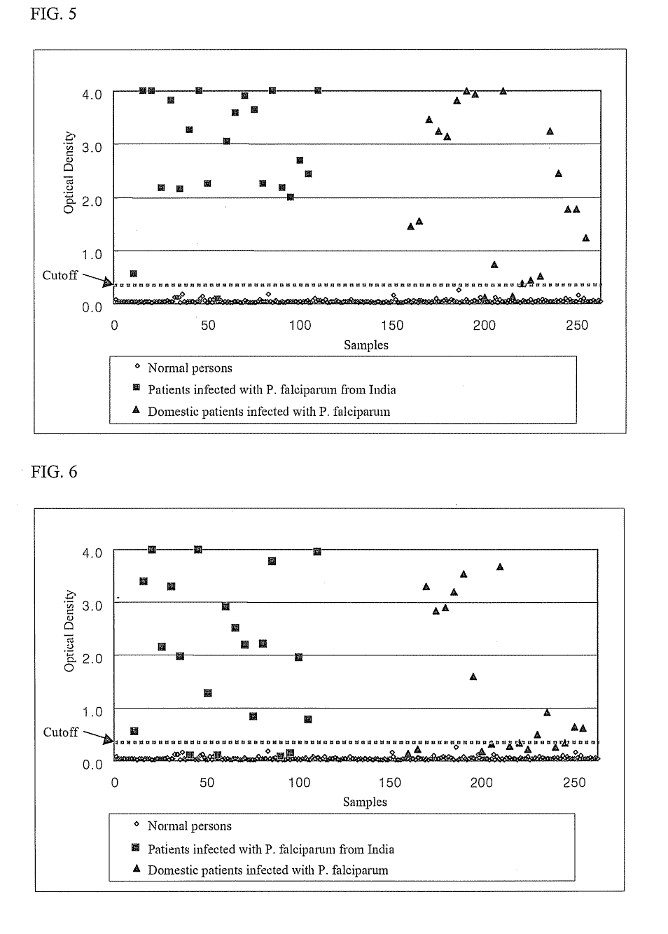

Upon examining and analyzing the results under the same conditions as in experimental example 1, samples of 264 sera from normal specimen were all tested negative. Upon examining 21 samples from Plasmodium falciparum-infected patients in India, it was found that 17 samples (81% sensitivity) were tested positive. For 20 samples from Korean patients infected with Plasmodium falciparum, it was found that 11 samples (55% sensitivity) were tested positive. The results thus obtained are graphically shown in FIG. 6.

Upon comparing FIG. 5 (experimental example 1) showing simultaneous detection results of antigen/antibody in accordance with the method of the present invention with FIG. 6 (comparative example 1) showing detection results of antibodies only, it can be seen that the method of the present invention exhibits higher sensitivity and specificity in detection of Plasmodium falciparum infection.

### Comparative Example 2

### Detection of Specific Antigens of Plasmodium falciparum Via Enzyme Immunoassay

For comparison with a method in accordance with the present invention, an enzyme immunoassay for determining specific antigens of Plasmodium falciparum only was carried out according to the following experiments.

Rabbit anti-HRP II prepared in example 3 was diluted to concentration of 5 to 10 μg/ml in 0.1 M carbonate buffer (pH 9.5) and 100 μl of the diluted solution was added into each well of a well plate. The well plate was sealed and incubated at room temperature for 18 hours such that antibodies added into each well were bound to the well plate. The solution remaining not adhered to the well plate was removed and then 300 μl of PBS containing 0.5% bovine serum albumin (BSA) was added into each well and incubated at room temperature for 6 hours. The solution remaining in the wells was removed and the well plate was dried and placed in a sealed bag, containing a desiccant, which was then stored in a low-temperature refrigerator at 4° C.

100 μl of sample diluent containing 1% BSA (bovine serum albumin), PBS (phosphate buffered saline) and Triton X-100 was added to each well and then 50 μl of sample was added and mixed well. The mixture in the well plate was reacted for 60 min in a incubator. After completion of reaction, the well plate was washed with 300 μl of PBS containing 0.05% Tween 20 five times.

Anti-HRP II-HRP conjugate prepared in example 4 was diluted with a casein/PBS diluent containing Tween 20. After completion of reaction, the well plate was washed with 0.05% Tween 20/PBS five times, and 100 μl of a substrate solution containing 100 μg/ml of TMB, 0.006% hydrogen peroxide and citric-phosphate buffer (pH 4.5) was added to each well and color development was carried out in a dark room for 30 min. 100 μl of stop solution (1N sulfuric acid) was added to each well to terminate color development reaction, and optical density (OD) at 450 nm was measured with a 96-well plate reader, using 650 nm as a reference wavelength. Since the color developer, TMB, in the substrate solution was catalyzed by the HRP conjugates bound to the antibodies, thereby causing color development reaction, the presence or absence of malaria-specific antibodies and intensity of antibody presence were detected by measuring the degree of color development in terms of optical density. Samples used in the above experiments were the same as those of experimental example 1.

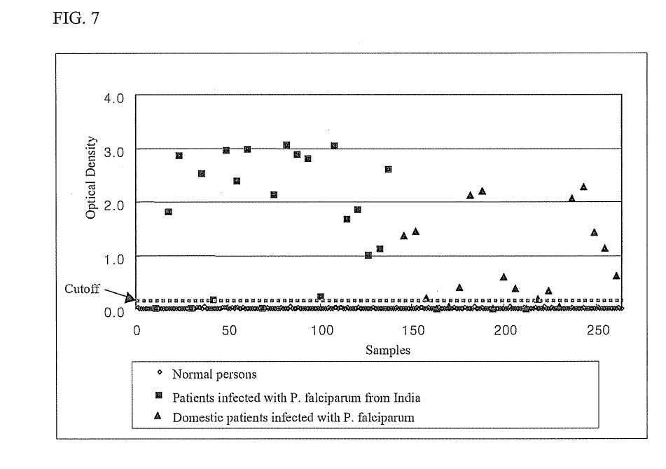

Upon examining and analyzing the results under the same conditions as in experimental example 1, samples of 264 sera from normal specimen, were all tested negative. Upon examining 21 samples from Plasmodium falciparum-infected patients in India, it was found that 18 samples (86% sensitivity) tested positive. For 20 samples from Korean patients infected with Plasmodium falciparum, it was found that 15 samples (75% sensitivity) tested positive. The results thus obtained are graphically shown in FIG. 7.

Upon comparing FIG. 5 (experimental example 1) showing simultaneous detection results of antigen/antibody in accordance with the method of the present invention with FIG. 7 (comparative example 2) showing detection results of antigen only, it can be seen that the method of the present invention exhibits higher sensitivity and specificity in detection of Plasmodium falciparum infection.

Further, samples, for which Plasmodium falciparum infection had not been confirmed or could not been convinced by the method of detecting antigens only (comparative example 1) and the method of detecting antibodies only (comparative example 2), were clearly confirmed to be Plasmodium falciparum infection by simultaneous detection of antigen/antibody in accordance with the method of the present invention. Therefore, it was demonstrated that the method in accordance with the present invention exerts significant effects surpassing simple combination of such prior arts.

### INDUSTRIAL APPLICABILITY

In accordance with the immunoassay and assay device of the present invention, it is possible to achieve simultaneous detection of antigens and antibodies specific to malaria (Plasmodium falciparum). Therefore, the present invention enables specific detection of antigens and/or antibodies of Plasmodium falciparum in patients with manifested malaria-symptoms as well as in carriers. In addition, the present invention exhibits very high specificity and sensitivity because malaria-specific antibodies are not detected in normal specimen and can also be efficiently employed in samples at the early stage of malaria infection that is difficult to detect via conventional arts. Further, due to the capability to use sera and blood plasma instead of whole blood, the present invention can be employed in a very suitable form for large-scale examination such as blood screening.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

What is claimed is:

1. An immunoassay for determining the presence of specific antigens and/or antibodies via labels in conjugates bound to the specific antigens and/or antibodies in a sample, comprising:
immobilizing a specific antigen and antibody of Plasmodium falciparum on solid phase;
adding a sample to induce the specific antigen and antibody reaction; and
adding a conjugate of the antigen and label (“antigen conjugate”) and a conjugate of the antibody and label (“antibody conjugate”), separately prepared, so as to induce binding of at least one of the conjugates.

2. The immunoassay according to claim 1, wherein the specific antigen is selected from the group consisting of a heat-shock protein 70 (HSP 70), merozoite surface protein (MSP) and glycophorin binding protein 130 (GBP 130) of Plasmodium falciparum and any combination thereof.

3. The immunoassay according to claim 1, wherein the specific antibody is an anti-histidine-rich protein II (anti-HRP II), and is a monoclonal or polyclonal antibody derived from animals or an antibody derived from genetic recombination.

4. The immunoassay according to claim 1, wherein HSP 70, MSP and GBP 130 are used in combination as the specific antigen, and the anti-BRP II is used as the specific antibody.

5. The immunoassay according to claim 1, wherein the label is selected from the group consisting of horseradish peroxidase (HRP), alkaline phosphatase, β-galactosidase, colloidal gold, fluorescent dye and any combination thereof.

6. The immunoassay according to claim 1, wherein binding of the conjugates to antigens and/or antibodies in the sample is confirmed by addition of a substrate that is catalyzed by the label in the conjugated and determination of whether the substrate is catalyzed or not.

7. The immunoassay according to claim 1, wherein the solid phase is a plastic, membrane, glass, or metallic support.

8. All assay device for conducting the immunoassay of claim 1, comprising:
a solid phase having a specific antigen and specific antibody of Plasmodium falciparum immobilized on the surface thereof; and
a conjugate of the antigen and a label (“antigen conjugate”) and a conjugate of the antibody and a label (“antibody conjugate”).


SEQUENCE LISTING (16 sequences)

SEQ ID NO: 1 - DNA, 29 bp, Artificial Sequence
  Pf-HSP70-NdeI (Primer)
ggatcccatatggctagtgcaaaaggttc

SEQ ID NO: 2 - DNA, 36 bp, Artificial Sequence
  Pf-HSP70-XhoI (Primer)
ggatccctcgagttaatcaacttcttcaactgttgg

SEQ ID NO: 3 - DNA, 30 bp, Artificial Sequence
  HSP70-SphI-5′ (Primer)
aatggtaaagaagcatgcagatcaattaac

SEQ ID NO: 4 - DNA, 30 bp, Artificial Sequence
  HSP70-SphI-3′ (Primer)
gttaattgatctgcatgcttctttaccatt

SEQ ID NO: 5 - DNA, 32 bp, Artificial Sequence
  Pf-GBP130-NdeI (Primer)
gattatcatatgagagaaagcagaattttagc

SEQ ID NO: 6 - DNA, 30 bp, Artificial Sequence
  Pf-G130BamHI-5′ (Primer)
agagaatatgccgcggatccagaatatcgt

SEQ ID NO: 7 - DNA, 30 bp, Artificial Sequence
  Pf-G130BamHI-3′ (Primer)
acgatattctggatccgcggcatattctct

SEQ ID NO: 8 - DNA, 33 bp, Artificial Sequence
  Pf-GBP130-XhoI (Primer)
ggatccctcgagttatgcttcgttattatcagc

SEQ ID NO: 9 - DNA, 35 bp, Artificial Sequence
  Pf-MSP19-NdeI (Primer)
gcaacacatatggcagtaactccttccgtaattga

SEQ ID NO: 10 - DNA, 36 bp, Artificial Sequence
  Pf-MSP19-XhoI (Primer)
gtgttgctcgagttataacataccttgcaagtttcc

SEQ ID NO: 11 - DNA, 33 bp, Artificial Sequence
  Pf-HRP II-NdeI (Primer)
ttgttacatatgagcaaaaatgcaaaaggactt

SEQ ID NO: 12 - DNA, 33 bp, Artificial Sequence
  Pf-HRP II-XhoI (Primer)
ataaatctcgagttaatggcgtaggcaatgtgt

SEQ ID NO: 13 - protein, 681 aa, Artificial Sequence
  Plasmodium falciparum′s agtigen (Heat-shock protein 70)
MASAKGSKPNLPESNIAIGIDLGTTYSCVGVWRNENVDIIANDQGNRTTPSYVAFTDTERLIGDAAKNQVARNPENTVFDAKRLIGRKFTESSVQSDMKHWPFTVKSGVDDKPMIEVTYQGEKKLFHPEEISSMVLQKMKENAEAFLGKSIKNAVITVPAYFNDSQRQATKDAGTIAGLNVMRIINEPTAAAIAYGLHKKGKGEKNILIFDLGGGTFDVSLLTIEDGIFEVKATAGDTHLGGEDFDNRLVNFCVEDFKRKNRGKDLSKNSRALRRLRTQCERAKRTLSSSTQATIEIDSLFEGIDYSVTVSRARFEELCIDYFRDTLIPVEKVLKDAMMDKKSVHEVVLVGGSTRIPKIQTLIKEFFNGKEACRSINPDEAVAYGAAVQAAILSGDQSNAVQDLLLLDVCSLSLGLETAGGVMTKLIERNTTIPAKKSQIFTTYADNQPGVLIQVYEGERALTKDNNLLGKFHLDGIPPAPRKVPQIEVTFDIDANGILNVTAVEKSTGKQNHITITNDKGRLSQDEIDRMVNDAEKYKAEDEENRKRIEARNSLENYCYGVKSSLVDQKIKEKLQPAEIETCMKTITTILEWLEKNQLAGKDEYEAKQKEAESVCAPIMSKIYQDAAGAAGGMPGGMPGGMPGGMPGGMPGGMNFPGGMPGAGMPGNAPAGSGPTVEEVD

SEQ ID NO: 14 - protein, 125 aa, Artificial Sequence
  Plasmodium falciparum′s agtigen (Merozoit surface protein)
HHHHHHSTGMVFENLAKTVLSNLLDGNLQGMLNISQHQCVKKQCPQNSGCFRHLDEREECKCLLNYKQEGDKCVENPNPTCNENNGGCDADAKCTEEDSGSNGKKITCECTKPDSYPLFDGIFCS

SEQ ID NO: 15 - protein, 219 aa, Artificial Sequence
  Plasmodium falciparum′s agtigen (Glycophorin binding protein 130)
HHHHHHSSGLVPRGSHMETRESRILAEGEDTCARKEKTTLRKSKQKTSTRTVATQTKKDEENKSVVTEEQKVESDSEKQKRTKKVVKKQINIGDTENQKEGKNVKKVIKKEKKKEESGKPEENKHANEASKKQEPKASKVSQKPSTSTRSNNEVKIRAASNQETLTSADPEGQIMETREYAADPEYRKHLEVFHKILTNTDPNDEVERQNADNNEASTP

SEQ ID NO: 16 - protein, 267 aa, Artificial Sequence
  Plasmodium falciparum′s agtigen (Histidine rich protein II)
DAHHAHHVADAHHAHHVADAHHAHHVADAHHAHHVADAHHAHHAADAHHAHHAAYAHHAHHAADAHHATDAHHAADAHHAADAHHAADAHHAHHAADAHHAHHAADAHHAHHAADAHHAHHAADAHHAHHAADAHHAHHAAYAHHAHHASDAHHAHHAADAHHAAYAHHAHHAADAHHAADAHHATDAHHAADAHHAADAHHATDAHHAADAHHAADAHHAAAHHATDAHHAAAHHATDAHHAAAHHATDAHHAAAHHEAATHCLRH